(19) United States
(12) Patent Application Publication
(10) Pub. No.: US 2009/0216114 A1
(43) Pub. Date: Aug. 27, 2009

(54) METHOD AND DEVICE FOR GUIDING A SURGICAL TOOL IN A BODY, ASSISTED BY A MEDICAL IMAGING DEVICE

(76) Inventors: Sebastien GORGES, Paris (FR); Yves TROUSSET, Palaiseau (FR); Regis VAILLANT, Bures Sur Yvette (FR)

Correspondence Address: General Electric Company;GE Global Patent Operation, PO Box 861, 2 Corporate Drive, Suite 648, Shelton, CT, 06484, US

(21) Appl. No.: 12/369,667

(22) Filed: Feb. 11, 2009

(30) Foreign Application Priority Data
Feb. 21, 2008 (FR) 0851115

(51) Int. Cl. A61B 5/05

(52) U.S. Cl. 600/425

(57) ABSTRACT

A method and device for real time navigation of a surgical tool handled by an operator in a region of interest of a body itself subject to at least one physiological movement.

### CROSS-REFERENCE TO RELATED APPLICATIONS

This application claims priority under 35 U.S.C. §119(a)-(d) or (f) to prior-filed, co-pending French patent application serial number 0851115, filed on Feb. 21, 2008, which is hereby incorporated by reference in its entirety.

### STATEMENT REGARDING FEDERALLY SPONSORED RESEARCH OR DEVELOPMENT

Not Applicable

### NAMES OF PARTIES TO A JOINT RESEARCH AGREEMENT

Not Applicable

### BACKGROUND OF THE INVENTION

1. Field of the Invention

The field of the present invention relates to a method and a device for guiding a surgical tool in the body of a patient, assisted by a medical imaging device, said patient being positioned on a table between an X-ray source and an image receiver of said medical imaging device.

2. Description of Related Art

In the field of no-invasive medical operations, the introduction of a surgical tool into an organ of a patient, such as a catheter for example in the vascular system right up a region of interest to be examined and/or to be treated is well known.

In this type of non-invasive operation, the position of the catheter relatively to the vascular system of the patient needs to be known in real time with the highest possible accuracy.

For this purpose, it is customary to use either navigation from fluoroscopic images, i.e. radiological images at low doses and in two dimensions, or an electromagnetic position sensor and a system for localizing said sensor.

In order to guide a surgical tool into an organ, fluoroscopic images, i.e. radiological images at low doses, acquired in real time by a radiographic device are frequently used. This type of device conventionally consists of a digital image receiver, an X-ray source emitting X-rays on the image receiver, said image receiver and X-ray source being respectively positioned at the ends of a C- or U-shaped arm, and said patient being positioned on a mobile table extending between the X-ray source and the image receiver. With these fluoroscopic images acquired in real time, the vascular system and the catheter in the region of interest may be viewed simultaneously.

However, this type of method has several important limitations. Taking into account the low X-ray dose used for acquiring these fluoroscopic images, the latter have low quality. For example, these fluoroscopic images do not provide any information in three dimensions, the operator having to mentally reconstruct a 3D or 2D representation of the organs of the patient and of the surgical tool.

In order to find a remedy to these drawbacks, 2D and 3D navigation techniques have already been devised, from images acquired prior to the operation. A 2D or 3D representation of the region of interest, such as for example the vascular system, is determined beforehand from images acquired by any imaging device well known to one skilled in the art, and then the position and the 2D or respectively 3D orientation of the surgical tool which is measured in real time by an electromagnetic sensor, are integrated in real time into the 2D or 3D static representation of the region of interest.

The guiding method therefore requires beforehand the determination of a 2D or 3D model of the region of interest of the patient; i.e. the region in which the tool navigates during the surgical operation. In order to obtain this model, any 2D or 3D imaging and reconstruction methods known to one skilled in the art may be used. For example, the 2D or 3D model of the region of interest of the patient may be obtained by a tomography method allowing acquisition of a portion of the patient per section and/or by a biplanar scanner allowing simultaneous acquisition of 2D images under two different angles and/or by a magnetic resonance imaging system and/or by an ultrasonic imaging system, and by applying adequate reconstruction algorithms known to one skilled in the art. Acquisition of the images is performed before the surgical operation, and then 2D or 3D images are stored either in the reconstructed form or in the form of images to be reconstructed with adequate reconstruction algorithms.

This type of method nevertheless has the drawback of not taking into account movements and deformations of the vascular system, more particularly at the breast and the heart, mainly induced by breathing. Thus, at a given instant, the form and the position of the static 3D representation of the vascular system frequently differs from the shape and the real position of said vascular system, inducing a deviation of the position and of the orientation of the surgical tool in the 3D representation of the vascular system at this given instant. Such a deviation is likely to be seriously detrimental to the success of the operation.

In order to determine and then compensate the movements and deformations of the organs of the region of interest, many methods are known which enhance the 3D navigation method.

A first method consists of placing an electromagnetic reference sensor on or close to the organ or on the skin of the patient at the region of interest, such as the breastbone of the patient for example, in order to determine the movement of the organ in the region of interest. The displacement determined by the reference sensor is then used for compensating the movements induced by breathing. A significant step of this method is the calibration of the transfer function between the movements of the reference sensor and the movements of the organ of the region of interest.

Such a method is notably described in the publications <<Holger Timinger et al, Physics in Medicine and Biology, (2004) PHILLIPS>>, <<So Zhang H, Banovac F, Glossop N, Cleary K, MICCAI, (2005) TRAXTA>> and <<Lo Bradford J. Wood, Journal of vascular and interventional Radiology (2005)>> and in the American Patent Application US 2005/00586177 and in the American U.S. Pat. No. 6,473,635.

A second method consists of using an additional breathing sensor, such as a breathing belt or a spirometer for example, in order to determine the breathing phase during the operating procedure. Knowing the breathing phase, the movements induced by breathing are concentrated by a deformation model. The parameters of the deformation model are calibrated at the beginning of the operating procedure and may possibly be updated during the navigation procedure.

This type of method is notably described in the publication <<Holger Timinger et al, Physics in Medicine and Biology, (2007) PHILLIPS>> and the American Patent Applications US 2007/0135713 and US 2003/220557.

All these methods have the drawback of requiring calibration before the beginning of the surgical operation and/or during the surgical operation, the practitioner having to cease navigation, i.e. his/her intervention, in order to proceed with calibration, which is a significant limitation in a clinical context.

### BRIEF SUMMARY OF THE INVENTION

Embodiments of the invention attempt to find a remedy to these drawbacks by proposing a method and a device for guiding a surgical tool in a body with which the movements of the organs in the region of interest due to the breathing of the patient may be compensated, with a simple and not very expensive design and not requiring a calibration operation by a user.

For this purpose and according to one embodiment of the invention, a method for real time navigation of a surgical tool handled by an operator in a region of interest of a body itself subject to at least one physiological movement is proposed. The method may comprise at least acquiring images of at least the region of interest by means of a medical imaging device; constructing a static 2D or 3D modelled representation of the region of interest by means of an image processing device; determining in real time the position of the surgical tool during the operation, in at least two dimensions of the region of interest subject to the physiological movement; compensating the position of the surgical tool or the static 2D or 3D modelled representation of the region of interest relatively to the physiological movement by means of a pre-established model for compensating the physiological movement or transfer function, and for viewing; and combining the static 2D or 3D modelled representation of the region of interest and the compensated position of the surgical tool or the compensated static 2D or 3D modelled representation of the region of interest and the position of the surgical tool. This method is remarkable in that the steps for compensating the physiological movement comprise at least the following steps for recording in real time a signal representing the movement of the surgical tool: detecting the physiological movement from the recorded signal representing the movement of the surgical tool; determining new parameters of the deformation model of the region of interest and/or the transfer function according to the detected physiological movement; and updating the deformation model of the region of interest and/or the transfer function with the new determined parameters.

The detection of the physiological movement from the recorded signal representing the movement of the surgical tool includes at least the following steps for determining a phase when the handling of the surgical tool by the operator is stopped, and determining the physiological movement in the phase when the handling of the surgical tool by the operator is stopped.

According to an alternative embodiment of the method, the detection of the physiological movement from the recorded signal representing the movement of the surgical tool includes at least the following steps for frequency breakdown of the recorded signal, and for determining the physiological movement from the achieved frequency breakdown.

Moreover, the method includes a step for determining the movement of the region of interest due to the breathing of the patient.

Said step for determining the movement due to the breathing of the patient includes at least the following steps: positioning a position sensor on the breastbone of the patient; recording the movements of the position sensor induced by the breathing of said patient; and determining the breathing phase from the movements of the position sensor.

Another embodiment of the invention provides an apparatus that may comprise: at least one device for acquiring images from at least the region of interest by means of a medical imaging device; a device for building a static 2D or 3D modelled representation of the region of interest by means of an image processing device; a device for determining in real time the position of the surgical tool during the operation in at least two dimensions of the region of interest subject to the physiological movement; a device for compensating the position of the surgical tool or of the static 2D or 3D modelled representation of the region of interest relatively to the physiological movement by means of a pre-established model for compensating the physiological movement or transfer function; and a viewing device combining the static 2D or 3D modelled representation of the region of interest and the compensating position of the surgical tool or the compensated static 2D or 3D modelled representation of the region of interest and the position of the surgical tool. The apparatus is remarkable in that the device for compensating the physiological movement comprises at least: a device for recording in real time a signal representing the movement of the surgical tool; device for detecting the physiological movement from the recorded signal representing the movement of the surgical tool, a device for determining new parameters of the deformation model of the region of interest and/or the transfer function depending on the detected physiological movement; and a device for updating the deformation model of the region of interest and/or the transfer function with the new determined parameters.

Said device for detecting the physiological movement from the recorded signal representing the movement of the surgical tool includes at least one device for determining a phase when the handling of the surgical tool by the operator is stopped, and a device for determining the physiological movement during the phase when the handling of the surgical tool by the operator is stopped.

According to an alternative embodiment of the apparatus, said device for detecting the physiological movement from the recorded signal representing the movement of the surgical tool includes at least one device for frequency breakdown of the recorded signal, and a device for determining the physiological movement from the achieved frequency breakdown.

Moreover, the apparatus may include a device for determining the breathing phase.

Said device for determining the breathing phase includes at least one position sensor, such as an electromagnetic sensor placed on the breastbone of the patient; and/or a breathing phase sensor, a breathing belt including a spirometer for example; and a device for breathing modelling, a so-called transfer function.

Said position sensor may be an electromagnetic sensor placed on the breastbone of the patient.

Further, said breathing phase sensor may be a breathing belt including a spirometer placed on the breastbone of the patient.

### BRIEF DESCRIPTION OF THE DRAWINGS

Other advantages and characteristics will become better apparent from the description which follows of several alternative embodiments, given as non-limiting examples of the method and device for guiding a surgical tool in a body, from the appended drawings wherein:

FIG. 1 is a schematic perspective view of an imaging device according to the invention;

FIG. 2 is a schematic illustration of the acquisition device of the imaging device according to the invention;

FIG. 3A is a schematic illustration of the algorithm for determining the physiological movements of the region of interest of the acquisition device of the imaging device according to the invention;

FIG. 3B is a schematic illustration of an alternative embodiment of the algorithm for determining physiological movements of the region of interest of the acquisition device of the imaging device according to the invention; and

FIG. 4 is a flowchart of the various steps of the method for guiding a surgical tool in a body assisted by a medical imaging device according to the invention

### DETAILED DESCRIPTION OF THE INVENTION

The method for guiding a surgical tool in a body assisted by a medical imaging device according to the invention of the X-ray type will be described hereafter; however, it is quite obvious that the guiding method according to the invention may be applied by a medical imaging device of the magnetic resonance type, or by any other medical imaging device well known to one skilled in the art, equipped with means according to the invention without however departing from the scope of the invention.

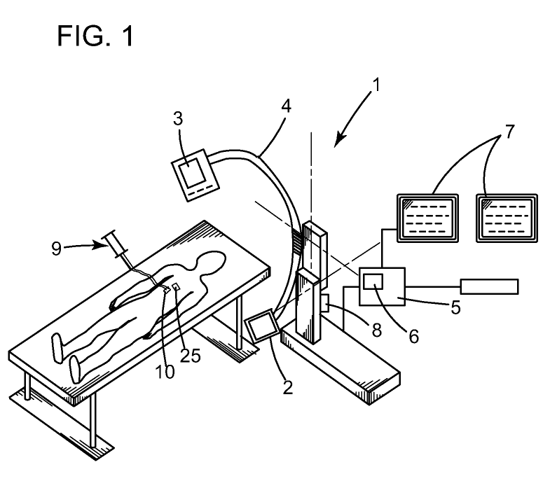

With reference to FIG. 1, the X-ray imaging apparatus 1 according to an embodiment of the invention, includes a digital image receiver 2, of an X-ray source 3 emitting X-rays on the image receiver 2, said image receiver 2 and the X-ray source 3 being respectively positioned at the ends of an arm in the shape of a C or U for example.

The imaging apparatus comprises monitoring means 5 connected to an acquisition device 6 and viewing means 7, said viewing means 7 usually consisting in a screen.

Further, the medical imaging apparatus includes a system 8 for determining the 3D position and orientation of a surgical tool 9, such as a catheter for example, provided with a position sensor 10, said system 8 being fixed, e.g., firmly attached for example to the medical imaging device and connected to the acquisition device 6.

The sensor 10 is an electromagnetic sensor, of a kind well known to one skilled in the art.

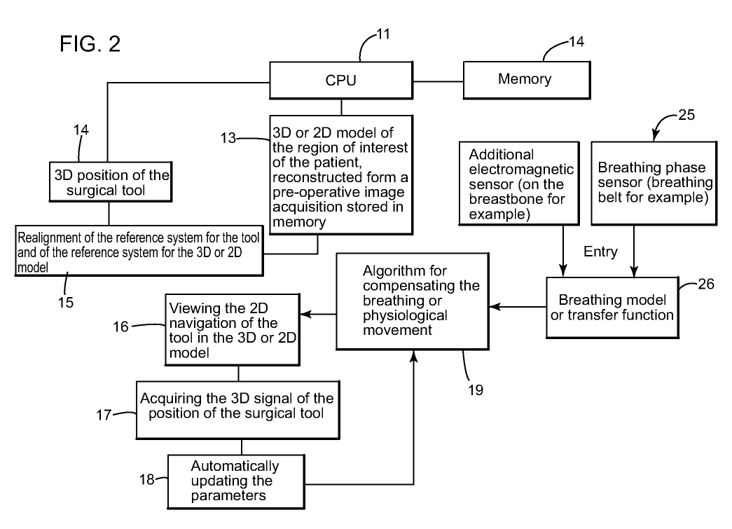

With reference to FIG. 2, the acquisition device 6 includes a computing unit 11, a memory 12 and a device 13 for constructing a static 2D or 3D modelled representation of the region of interest by means of an image processing device, such as a 2D or 3D representation of the vascular system of the region of interest. This device 13 may include an algorithm recorded in the memory 12 for example, which determines the 2D or 3D representation of the organ of the patient from images acquired prior to the operating phase by the medical imaging device. For example, the 2D or 3D model of the region of interest of the patient may be obtained by a tomography method allowing acquisition of a portion of the patient per section and/or by a biplanar scanner allowing simultaneous acquisition of two 2D images under two different angles and/or by a magnetic resonance imaging system and/or by an ultrasonic imaging system and application of adequate reconstruction algorithms known to one skilled in the art. The acquisition of the images is completed before the surgical operation, and then the 2D or 3D images are stored in the memory 12 either in the reconstructed form or in the form of images to be reconstructed with the adequate reconstruction algorithms.

The term “algorithm” refers to a computer program suitable of executing a succession of computations or steps within a determined time.

The acquisition device 6 also includes an algorithm 14 for determining in real time the 2D or 3D position of the surgical tool from the system 8, for determining the 2D or 3D position and orientation of a surgical tool and a device 15 for realigning the reference system of the tool and the reference system of the 2D or 3D model 13.

The apparatus moreover includes an algorithm 16 for viewing, combining the static 2D or 3D modelled representation of the region of interest and the compensated position of the surgical tool or the compensated static 2D or 3D modelled representation and the position of the surgical tool, said images being generated in real time and viewed on the viewing screen 7. An algorithm 17 for recording in real time a signal representing the movement of the surgical tool 9 provides the required information to an algorithm 18 for detecting the physiological movement from the recorded signal representing the movement of the surgical tool, and determining the new parameters of the deformation model of the region of interest and/or of the transfer function according to the detected physiological movement, and updating the deformation model of the region of interest and/or the transfer function with the new determined parameters.

Moreover, the apparatus includes an algorithm 19 for compensating the position of the surgical tool or the static 2D or 3D modelled representation of the region of interest relatively to the physiological movement by means of a pre-established model for compensating the physiological movement or transfer function.

It will be noted that the compensation model and/or the transfer function are either pre-recorded in the memory 12, the user selecting in a data base the suitable model and/or the transfer function depending on the localization of the region of interest for example, or determined prior to the surgical operation or during the latter.

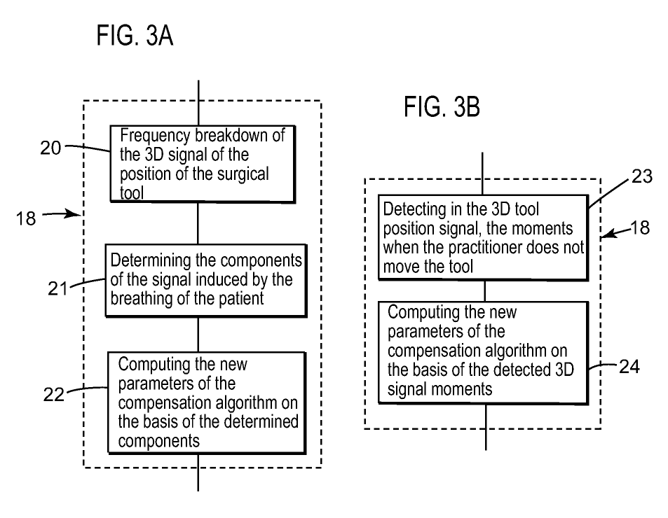

According to a first alternative embodiment of the apparatus, with reference to FIG. 3B, the algorithm 18 includes an algorithm 23 for determining a phase when the handling of the surgical tool by the operator is stopped from a recorded signal representing the movement of the surgical tool and an algorithm 24 for determining the physiological movement in the phase when the handling of the surgical tool by the operator is stopped and for computing new parameters of the compensation algorithm and/or of the transfer function from the physiological movement determined by the algorithm 23.

Thus, the acquisition device 6 determines that the surgical tool 9 is no longer handled when the movements of the surgical tool 9 are for example globally periodic. Said acquisition device 6 then determines the movements of the organ then it calibrates and/or updates the deformation model of the organ of the region of interest, and/or the transfer function depending on said physiological movements when a user again displaces the surgical tool 9. In this way, the 2D or 3D representation into which the 3D position and orientation of the surgical tool 9 is integrated, displayed in real time, is automatically calibrated whenever the user has a break in the handling of the surgical tool 9.

According to a second alternative embodiment of the apparatus, with reference to FIG. 3A, said algorithm 18 includes at least one algorithm 20 for frequency breakdown of the recorded signal representing the position of the surgical tool, such as a Fourier breakdown algorithm well known to one skilled in the art for example, an algorithm 21 for determining the physiological movement from the frequency breakdown and an algorithm 22 which determines new parameters of the deformation model of the region of interest and/or the transfer function depending on the physiological movement determined by the algorithm 21.

In this particular exemplary embodiment of the invention, the physiological movements are determined regardless of whether the surgical tool 9 is displaced or not. In this way, the deformation model for the organ of the region of interest will be calibrated and/or updated depending on the frequency breakdown in real time or at regular intervals, and the 3D position of the surgical tool 9 integrated into the static 3D modelled representation of the vascular system will either be compensated in real time or at regular intervals.

Accessorily, with reference to FIGS. 1 and 2, the apparatus includes a device independent of the determination of the movement of the region of interest due to the breathing of the patient. This device includes at least one position sensor 25, such as an electromagnetic sensor placed on the breastbone of the patient and/or a breathing phase sensor, a breathing belt including a spirometer for example, and a breathing modelling algorithm 26, a so-called transfer function.

With this device, it is possible to determine at each instant the movement of the region of interest due to the breathing of the patient and to carry out the appropriate correction of the deformation model and/or of the transfer function at each instant. For this purpose, an algorithm for separating the cyclic movement due to breathing and the movement of the surgical tool may be used without using any frequency breakdown, the algorithm being able to extract from the acquired signal of the position of the surgical tool, a periodic component, the phase and the period of which are determined by the device for determining the breathing phase.

Accessorily, it will be noted that the practitioner may if need be use fluoroscopic images acquired by the medical imaging device 1 for example, ultrasonic images, endoscopic images, etc. in order to make sure that the compensation of the physiological movements such as the movement due to breathing, is properly calibrated in the static 2D or 3D modelled representation of the region of interest which he/she views on the screens 7.

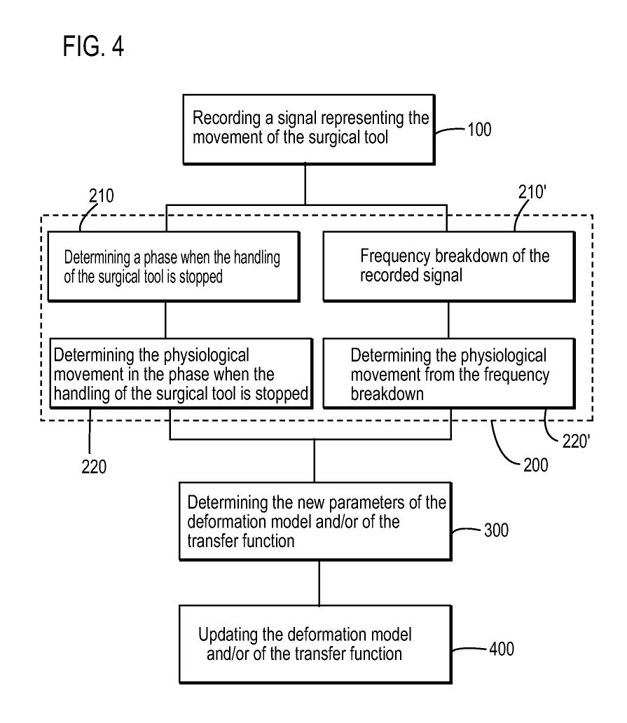

The operation of the apparatus will now be explained with reference to FIG. 4.

In a first step 100, a signal representing the movement of the surgical tool 9 is recorded.

In a step 200, the physiological movement of the region of interest is detected from the recorded signal representing the movement of the surgical tool.

According to a first alternative embodiment, the step 200 for detecting the physiological movement from the recorded signal representing the movement of the surgical tool, includes a step 210 for determining a phase when the handling of the surgical tool is stopped, and then a step 220 for determining the physiological movement of the region of interest during the phase when the handling of the surgical tool is stopped.

According to a second alternative embodiment, the step 200 for detecting the physiological movement from the recorded signal representing the movement of the surgical tool, includes a step 210′ for frequency breakdown of the recorded signal and then a step 220′ for determining the physiological movement of the region of interest from the frequency breakdown achieved beforehand.

The new parameters of the deformation model and/or of the transfer function, are then determined in a step 300, and then the deformation model and/or the transfer function are updated in a step 400.

Finally, it is understood that the examples which have just been given are only particular illustrations of the method and device for guiding a surgical tool in a body, by no means limiting as to the scope of the invention, which is defined the appended claims.

1.-12. (canceled)

13. A method for real-time navigation of a surgical tool handled by an operator in a region of interest of a body itself subject to a physiological movement, the method comprising:
acquiring images of at least the region of interest using a medical imaging device;
constructing a static 2-D or 3-D modeled representation of the region of interest using an image processing device;
determining in real time a position of the surgical tool during operation, in at least two dimensions of the region of interest being subject to the physiological movement;
compensating for the position of the surgical tool or the static 2-D or 3-D modeled representation of the region of interest relative to the physiological movement using a pre-established deformation model or a transfer function; and
combining the static 2-D or 3-D modeled representation of the region of interest and a compensated position of the surgical tool with a compensated static 2-D or 3-D modeled representation of the region of interest in the position of the surgical tool,
wherein compensating for position of the surgical tool further comprises:
recording in real time a signal representing a movement of the surgical tool;
detecting the physiological movement from the recorded signal representing the movement of the surgical tool;
determining new parameters of a deformation model of the region of interest and/or the transfer function depending on the detected physiological movement; and
updating the deformation model of the region of interest and/or the transfer function with the new determined parameters,
wherein the detecting of the physiological movement from the recorded signal representing the movement of the surgical tool comprises:
breaking down a frequency of the recorded signal; and
determining the physiological movement from the achieved frequency breakdown.

14. The method of claim 13, further comprising:
determining movement of the region of interest due to a breathing of a patient.

15. The method of claim 14, wherein the step for determining movement of the region of interest due to the breathing of the patient further comprises:
recording movements of a position sensor positioned proximate a breast of the patient that are induced by the breathing of the patient; and
determining a breathing phase from the movements of the position sensor.

16. An apparatus, comprising:
a device configured to provide real-time navigation of a surgical tool handled by an operator in a region of interest in a body itself subject to a physiological movement;
a medical imaging device configured to acquire an image of the region of interest;
an image processing device configured to construct a static 2D or 3D modeled representation of the region of interest using the acquired image of the region of interest;
a device configured to determine in real-time a position of the surgical tool during operation in at least two dimensions of the region of interest;
a device configured to compensate a position of the surgical tool relative to a detected physiological movement using a pre-established deformation model or transfer function and comprising:
a device configured to record in real-time a signal representative of a movement of the surgical tool;
a device configured to detect the physiological movement from the recorded signal and comprising:
a device configured to breakdown a frequency of the recorded signal, and
a device configured to determine the physiological movement from the frequency breakdown;
a device configured to determine new parameters of the pre-established deformation model and/or transfer function; and
a device configured to update the pre-established deformation model and/or transfer function with the new parameters.

17. The apparatus of claim 16, further comprising:
a device configured to determine a breathing phase of a patient.

18. The apparatus of claim 17, wherein the device configured to determine a breathing phase of a patient comprises:
a position sensor configured to be located proximate a breast of the patient;
a breathing phase sensor; and
a device configured to model the breathing phase.

19. The apparatus of claim 18, wherein the breathing phase sensor is a spirometer.
